(19) United States
(12) Patent Application Publication
(10) Pub. No.: US 2021/0228403 A1
(43) Pub. Date: Jul. 29, 2021

(54) DEVICE AND METHOD FOR NASAL BREATHING

(71) Applicant: Maedyn IP LLC, Casper, WY (US)

(72) Inventor: Jessi WARING, Casper, WY (US)

(21) Appl. No.: 16/752,725

(22) Filed: Jan. 27, 2020

(51) Int. Cl. A61F 5/56; A61N 1/05; A61B 5/0205; A61B 5/00; A61N 1/04

(52) U.S. Cl. CPC A61F 5/56; A61N 1/0546; A61N 1/0496; A61B 5/0205; A61B 5/4809; A61N 1/0548

(57) ABSTRACT

An embodiment herein relates to a wearable device comprising three strips of medical-grade, hypoallergenic adhesive wherein two of the strips lie above and below the mouth, and the third strip connects the first two and bridges the gap across the lips, keeping them closed. The strips are connected by hook and loop attachments, allowing them to be removed and reattached as needed when used. The wearable device may have additional features, included but not limited to being reusable, containing electrodes to stimulate muscles, various sensors, a handheld device, added ointment or skin-absorbed medication, a nasal dilator, different colors, and/or cloth padding for added comfort.

### FIELD OF THE INVENTION

This invention relates to the process of preparing an apparatus to promote nasal breathing. The invention is more particularly concerned with a process involving sealing the lips closed temporarily and reversibly in order to stop and resume oral activities as needed.

### BACKGROUND OF INVENTION

Sleep-disordered breathing is an umbrella term for several chronic conditions in which partial or complete cessation of breathing occurs many times throughout the night. Sleep-disordered breathing results in release of stress hormones with daytime sleepiness or fatigue that interferes with a person's ability to function and reduces quality of life. Symptoms may include snoring, pauses in breathing described by bed partners, and increased respiratory effort. Upper airway resistance syndrome, which is by far the most common form of sleep-disordered breathing, is associated with many other adverse health consequences, including an increased risk of death.

To be properly diagnosed, patients with suspected sleep-disordered breathing are often evaluated by a polysomnogram (sleep test), which measures approximately a dozen physiologic parameters during sleep. One of the most important measurements is breathing effort and its cessation during sleep. A breathing pause of 10 seconds or more is generally termed an apnea. Not surprisingly, apneas may be associated with oxygen desaturation (a decrease in blood oxygen) and other bodily responses, as the person struggles to breathe. These arousals may consist of flexing of muscles, including those of the airways, and change in the electrical activity of the brain as measured by an electroencephalogram (EEG). Arousals are complex phenomena that may involve discharges of brain chemicals of the adrenalin family, which may contribute to the health conditions associated with sleep apnea. Desaturation and arousals also occur with hypopnea (partial decrease in air flow). The Apnea-Hypopnea Index is the number of apneas and hypopneas that occur per hour of sleep and is an important measure of the severity of sleep apnea, along with the depth of denaturation.

A single-night polysomnogram in a sleep laboratory can accurately diagnose sleep apnea in most patients. With portable equipment, the diagnosis of sleep apnea is possible in the home setting, and this approach may provide improved access to sleep apnea diagnostic testing. Important indexes from sleep studies are the Apnea/Hypopnea Index (AHI) the Respiratory Disturbance Index (RDI) and oxygen saturations. AHI is a measure that indicates the severity of sleep apnea. It is the average number of apneas and hypopneas per hour of sleep. This is calculated by adding the total number of all apneas and hypopneas and then dividing them by the number of hours the patient spends sleeping. This measure represents the severity of sleep apnea including sleep disturbances and desaturations.

RDI is a measure of the severity of sleep apnea, including sleep disruptions and desaturations. Unlike the AHI, the RDI counts the number of arousals by respiratory effort. It is the average number of sleep disordered events that cause an arousal from sleep per hour of sleep. It is calculated by adding the number of apneas, hypopneas, and respiratory effort related arousals (RERAs) and dividing by the number of hours the patient spends asleep.

Estimates of the prevalence of sleep-disordered breathing vary widely, depending on the methodology. Conservatively, based on laboratory or portable home tests, 4 percent of men, 2 percent of women, and 2 percent of children ages 8 to 11 in the United States have been reported to have sleep-disordered breathing. Other surveys estimate that between 5 and 10 percent of the U.S. adult population have Obstructive Sleep Apnea (OSA); 7 percent have breathing pauses during sleep that put them at risk for more severe sleep events, and 23 to 59 percent snore. Unpublished data from a nationally representative sample of U.S. adults over age 20 show that the symptoms of sleep-disordered breathing (for example, snoring) are more likely to be reported by men than women. From 1980 to 1990, the number of office visits in the United States resulting in a diagnosis of sleep apnea increased from 108,000 to 1.3 million. Despite the increased awareness of sleep-disordered breathing, it has been suggested that 93 percent of women and 82 percent of men with signs and symptoms of moderate to severe sleep-disordered breathing remain undiagnosed.

Factors that have been identified in studies to increase the risk of developing sleep apnea include obesity, male gender, and some ethnic groups (African American, Asian, and Native American). A study of more than 6,400 patients with mild to moderate sleep-disordered breathing found an association between sleep-apnea severity as measured by the Apnea-Hypopnea Index (AHI) and coronary artery disease, heart failure, and stroke. Those with the highest AHI were one-and-a-half times more likely to have had a stroke and more than twice as likely to have heart failure than those with lowest AHI, even when adjusted for other known risk factors, including age, sex, race, body size, hypertension, smoking, and cholesterol levels.

The economic burden of sleep-disordered breathing is significant. Lack of adequate sleep at night for any reason leads to daytime somnolence, and habitual lack of restful sleep can lead to uncontrollable sleep attacks. Sleep-disordered breathing adversely affects daytime alertness and cognition and has been linked to occupational and driving impairment. Sleep apnea has also been shown to increase healthcare utilization. In any assessment of the economic burden of sleep apnea, there are two important considerations: 1) it is highly prevalent in the middle-aged work force, and 2) it contributes to other chronic health conditions, such as heart disease and diabetes, and increases the risk of having a stroke and being in an accident at work or in an automobile.

No single cause of sleep apnea has been identified, although an association with weight and neck size is well known. Causes may include nasal obstruction; mouth breathing; large tonsils (particularly in children); an underactive thyroid gland; the use of alcohol, tobacco, and sedatives; menopause in women; and higher levels of testosterone. Family history and genetic susceptibility studies show that a third of the total variability in sleep apnea severity in populations can be accounted for by heritability or genetic susceptibility. The bony and soft tissue structures of the face, as well as the heritability of obesity, are potential mechanisms by which genetics plays a role in sleep apnea.

A severe form of breathing disorder of sleep is OSA, noted above, which is characterized by recurrent narrowing or collapse of the back of the throat because of the loss of muscle tone that occurs during sleep. A less common form, central sleep apnea, is distinguished by cessation of breathing efforts during sleep. There is no struggle to breathe; the brain just does not send the proper breathing signals. Both result in repetitive events of insufficient air flow, oxygen absorption, and carbon dioxide exhalation. Reduction in blood oxygen levels may lead to a hormonal stress response by the body. This reaction may arouse, but not fully awaken, the sleeper, who repeats the events with the next period of sleep. If the cycle of arousals is repeated many times during the night, a cascade of stress-hormone release ensues, which is thought to be responsible for many of the adverse health consequences associated with sleep-disordered breathing. A very common form of increased effort to breath is upper airway resistance syndrome (UARS). This is a sleep-related breathing disorder in which repetitive increases in resistance to airflow in the upper airway lead to brief arousals (RERAs) and daytime fatigue. It is usually associated with loud snoring. Obstructive Sleep Apnea (OSA), characterized by apneas and hypopneas, may be totally absent. Although blood oxygen levels may be in the normal range, the patient can still have symptoms of Obstructive Sleep Apnea, e.g., excessive daytime sleepiness. This is a result of the stimulation of sleep arousals and their repetitive release of stress hormones.

The upper airway in patients with OSA is often smaller than normal. It may be narrowed by fat deposition in obese individuals or other structural factors, such as airway length, position of the jaw, or size of the tongue. A narrowed air passage can collapse more frequently and completely when the muscles of the throat, which keep the upper airway open during wakefulness, relax during sleep. Changes in body position and the reduced lung expansion that occur with sleep interact with these other factors and may lead to further upper airway vulnerability.

Experimental animal studies as well as observations in patients with central sleep apnea show that the brain centers responsible for the control of rhythmic respiratory muscle activity are more unstable compared to people without this disorder. Some individuals may have both obstructive and central sleep apnea. For children suffering from sleep apnea, surgical treatment with removal of tonsils (and adenoids) is the first choice. However, the long-term effects of this procedure on sleep-disordered breathing in these children are poorly understood.

There is considerable variation in the severity of sleep apnea from night to night, depending upon duration of sleep, body position, time spent in different stages of sleep, and other factors, such as alcohol consumption before going to bed. Alcohol and certain sleeping medications may cause deeper relaxation of the airways during sleep and a blunting of the sleeper's arousal response, thus allowing longer and more frequent apneas and greater desaturations.

Prevention of weight gain and obesity is critical for reducing the risk of developing clinically significant OSA. Appropriate evaluation and treatment of any nasal passage obstruction is important in reducing the collapsibility of the upper airway. Smoking cessation should be pursued by all patients. Avoiding alcohol and sedatives and developing better sleep hygiene may be helpful.

Physicians use the Apnea-Hypopnea Index (AHI) to assess the severity of sleep apnea based on the number of complete cessations of breathing (apnea) and partial obstructions (hypopnea). Although the Apnea-Hypopnea Index (AHI) is interpreted in the context of the patient's symptoms, age, and other medical conditions, an Apnea-Hypopnea Index (AHI) of more than 5 with symptoms is generally abnormal enough to warrant treatment. As the condition is usually chronic, in the absence of significant modification of a risk factor, the treatment prescribed should be used long term.

Treatments for OSA work by physically increasing the size of the upper airway. A very effective treatment is a continuous positive airway pressure (CPAP) device that delivers pressurized air to the upper airway, via a mask, splinting the airway open. However, the effectiveness of this treatment is often substantially reduced or nullified by inconsistent or inadequate use by patients. Professionally assisted adjustments of the mask size and type, the addition of humidity, and the treatment of nasal congestion and blockage may improve the ability to use this treatment.

There is no presently available effective and safe drug treatment for sleep apnea. External and intra nasal dilators improve snoring, but their efficacy in reducing sleep-disordered breathing has not been adequately shown by controlled trials. In certain patients, surgical treatment or dental devices may be effective, but more studies are needed.

Although breathing abnormalities that occur during wakefulness and sleep have been reported since the 1800s, the high prevalence of disordered breathing that occurs only during sleep was not recognized until 1993. The risk factors for sleep-disordered breathing and the high prevalence of sleep apnea, as well as the adverse health conditions associated with untreated sleep apnea, including increased mortality, have been identified by multiple large-scale observational studies. There is, however, an urgent need for large-scale clinical studies to determine the natural course and benefit of treatments on the longer-term health in people with all levels of sleep-disordered breathing, especially with regard to its severity, effect on cardiovascular health, and survival.

Given the remarkable rise of obesity and the high prevalence of diabetes today, it would also be important to learn the effects of these conditions on the course and treatment of sleep apnea. Intervention at early stages has the potential to become an effective prevention strategy. Confirmation of whether portable and home-based diagnostic monitoring and auto-adjusting therapeutic CPAP devices could adequately supplement formal laboratory-based evaluation, and, if so, in which populations, would lead to more cost-effective healthcare delivery. Studies thus far support the use of oral appliances in mild to moderate sleep-disordered breathing and the use of surgery primarily as adjunctive treatment for adults or in “CPAP failures.” Electrical stimulation of the nerves to activate the upper airway muscles and dilate the airway has been associated with beneficial effects on sleep-disordered breathing, but this approach needs further study to determine efficacy as well as the design of equipment for clinical use.

It is as yet not clear whether the candidate genes for sleep apnea (for example, the APOE epsilon gene) lead directly to sleep apnea or if these genes are linked to intermediate factors that increase the risk of sleep apnea via their effects on other factors, such as facial structure and obesity. Future studies involving analysis of multiple genes simultaneously in well-defined subgroups of persons with sleep apnea hold the promise for development of predictive models that will enable early diagnosis and intervention in the appropriate populations. A genetic approach also may lead to better understanding of the basic mechanisms of the condition, which is a prerequisite for the development of future therapies.

Though multiple potential solutions exist for promoting nasal breathing, one that is comfortable, easily applied, and effective is still a pressing issue. Given the importance of promoting nasal breathing during sleep, for reasons including but not limited to snoring, dry mouth, and sleep apnea, it is crucial that any device prepared must be suitable for daily, comfortable wear.

Currently, the preparation of nasal apparatuses preventing oral breathing features either adhesive strips of tape temporarily holding the lips together, or a facial mask that covers the mouth and nose. Though these devices are effective, they are not easily removable, and especially in the case of tape-based devices, they are not reusable once the adhesive is broken. The present embodiment overcomes these limitations while providing the same efficacy other devices have.

Examples of using tape or other sorts of adhesive are seen in U.S. Pat. No. 20160278973 U.S. Pat. No. 20160302961, and U.S. Pat. No. 20080053459, where a gentle adhesive is applied across the lips in order to keep them shut while the user sleeps. In U.S. Pat. No. 20160278973, the device described is made up an adhesive strip above and below the user's mouth, with a non-adhesive, low-friction strip over the user's mouth, allowing the user's lips to move freely in order to breathe, cough, or speak. U.S. Pat. No. 20160302961 describes a similar device, where an adhesive strip is placed over the lips, holding them shut, and incorporates a non-adhesive peripheral tab aiding the user in gently peeling off the device after use. U.S. Pat. No. 20080053459 describes a device where the lips are sealed to adhesive under a pocket created by two panels covering the mouth, effectively preventing the mouth from opening completely and taking in enough air to support snoring.

Examples of oral occlusion devices based on a mask pattern are seen in U.S. Pat. Nos. 9,962,513, 8,291,906, and U.S. Pat. No. 2007/0044803. U.S. Pat. No. 9,962,513 describes a mask assembly designed to correct sleep-disordered breathing where a mask provides pressurized air, attached to the user's face during sleep through headgear attached with hook and loop attachments. U.S. Pat. No. 8,291,906 describes a similar device where a mask adheres over the user's nose with both adhesive and a hook and loop attachment and engages the air way in order to correct breathing. U.S. Pat. No. 2007/0044803 describes another mask-style device where a respirator made up of a mask body and a nose clip covers the user's nose and mouth, attached by adjustable elastic straps.

In the aforementioned tape-based designs, they cannot easily be removed and reattached. Once the device is removed, the adhesive is not as effective and a new one must be used. If the user wants to drink, speak, cough, or perform any other activity with their mouth, they must either remove the entire device and use a new one, or perform that activity with the limited ability afforded to them by keeping the device on. The mask-based designs have limitations as well. While they can be reusable, they also have limiting mobility, and by way of their design cannot always facilitate comfortable sleeping in every position. The present embodiment overcomes these limitations while providing the same level of efficacy previous devices have shown.

### SUMMARY OF INVENTION

An embodiment relates a wearable device comprising an adhesive strip and a bridging strip, the adhesive strip comprising a medical-grade hypoallergenic adhesive that is configured to be adhered to a face of a human or an animal at locations above and below lips of the human or the animal, wherein the bridging strip is configured to be placed over the lips so as to bridge the adhesive strip and hold the lips shut via a hook and loop attachment, wherein the hook and loop attachment is configured to permit removing and reattaching of the bridging strip for a plurality of times without loss of ability to hold the lips shut, wherein the wearable device is configured to hold the lips shut and promote nasal breathing.

In an embodiment, the adhesive strip comprises a single piece of adhesive strip or a plurality of adhesive strips, and the adhesive strip has a first side and a second side, wherein the first side comprises the medical-grade hypoallergenic adhesive and the second side comprises at least a hook portion or a loop portion of the hook and loop attachment.

An embodiment relates to a wearable device comprising a plurality of adhesive strips and a bridging strip, each of the plurality of adhesive strips comprising a medical-grade hypoallergenic adhesive that is configured to be adhered to a face of a human or an animal at locations above and below lips of the human or the animal, wherein the bridging strip is configured to be placed over the lips so as to bridge the plurality of adhesive strips and hold the lips shut via a hook and loop attachment, wherein the wearable device is configured to hold the lips shut and promote nasal breathing.

In an embodiment, each of the plurality of adhesive strips has a first side and a second side, wherein the first side comprises the medical-grade hypoallergenic adhesive and the second side comprises at least a hook portion or a loop portion of the hook and loop attachment.

In an embodiment, the bridging strip comprises at least a hook portion or a loop portion of the hook and loop attachment.

The wearable device could further comprise an electrode embedded in the plurality of adhesive strips.

In an embodiment, the electrode is configured to electrically stimulate an orbicularis oris muscle to contract and close the lips.

The wearable device could further comprise a sensor embedded in the bridging strip.

In an embodiment, the sensor is configured to measure oxygen saturation, respiration, sleep, and pulse.

In an embodiment, at least one of the plurality of adhesive strips comprise a moisturizer, a medicated ointment, a medicated gel, a medicated coating.

In an embodiment, at least one of the plurality of adhesive strips comprise a nasal dilator strip.

In an embodiment, the plurality of adhesive strips have a color and/or a pattern.

In an embodiment, the color comprises clear or skin tone, and the pattern comprises a decorative pattern.

The wearable device could further comprise a padding over the plurality of adhesive strips.

An embodiment relates to a system comprising a wearable device and a handheld device, the wearable device comprising a plurality of adhesive strips, a bridging strip, an electrode embedded in the plurality of adhesive strips, and a sensor embedded in the bridging strip, wherein each of the plurality of adhesive strips comprises a medical-grade hypoallergenic adhesive that is configured to be adhered to a face of a human or an animal at locations above and below lips of the human or the animal, wherein the bridging strip is configured to be placed over the lips so as to bridge the plurality of adhesive strips and hold the lips shut via a hook and loop attachment, wherein the wearable device is configured to hold the lips shut, and wherein the handheld device is configured to control the electrode.

An embodiment relates to a method comprising obtaining a wearable device and attaching the wearable device to a face of a human or an animal, wherein the wearable device comprises a plurality of adhesive strips and a bridging strip, each of the plurality of adhesive strips comprising a medical-grade hypoallergenic adhesive that is configured to be adhered to the face of the human or the animal at locations above and below lips of the human or the animal, wherein the bridging strip is configured to be placed over the lips so as to bridge the plurality of adhesive strips and hold the lips shut via a hook and loop attachment, wherein the wearable device is configured to hold the lips shut and promote nasal breathing.

An embodiment relates to preparing a nasal breathing apparatus comprising three pieces of medical-grade hypoallergenic adhesive. One piece of adhesive is placed above the upper lip and under the nose. The second piece of adhesive is placed below the lower lip. The third piece of adhesive is placed across the lips as the user rolls their lips slightly inward, connecting the two aforementioned pieces by way of hook and loop attachments and keeping the lips “pinched” shut.

In one embodiment, the medical-grade hypoallergenic adhesive is in the form of tape.

In one embodiment, the first two pieces of adhesive measure approximately three and a half inches long by approximately half an inch wide, while the third piece of adhesive measures approximately three and a half inches long by approximately one inch wide.

In one embodiment, the hook and loop attachments are similar to VELCRO®

Alternate embodiments may include variations on the basic design in order to improve functionality in a specific outlying case and/or add additional features for greater efficacy or more information.

In an embodiment, the device is reusable, where adhesive hydrogel on the upper and lower strips may be taken off and reapplied without losing efficacy.

In an embodiment, the device includes small wireless electrodes to the strips above and below the lips to stimulate the orbicularis oris muscles to contract, closing the lips and encouraging nasal breathing. This feature may be turned on and off as desired in order to train lip closure and nasal breathing and reduce snoring.

In an embodiment, the device includes a pulse oximeter, respirations tracker, sleep time tracker, and/or pulse tracker attached to the reusable bridging piece to track and record data.

In an embodiment, the device includes a handheld device in order to adjust electrode stimulation in terms of force, frequency, and duration of the contraction, as well as tracking all vitals described above and providing alerts as needed.

In an embodiment, the adhesive directly in contact with the skin contains a medicated ointment, gel, or other substance to combat dry/cracked lips and/or provide additional comfort to the user.

In an embodiment, a nasal dilator strip is attached to further facilitate nasal breathing and combat snoring.

In an embodiment, the device is skin-toned, clear, or has a decorative pattern appealing to users, especially young users.

In an embodiment, the adhesive strips are covered with cloth padding in order to be more comfortable for back and side sleepers.

### BRIEF DESCRIPTION OF THE FIGURES

In the present disclosure, reference is made to the accompanying drawings, which form a part hereof. In the drawings, similar symbols typically identify similar components, unless context dictates otherwise. Various embodiments described in the detailed description, drawings, and claims are illustrative and not meant to be limiting. Other embodiments may be used, and other changes may be made, without departing from the spirit or scope of the subject matter presented herein. It will be understood that the aspects of the present disclosure, as generally described herein, and illustrated in the Figures, can be arranged, substituted, combined, separated, and designed in a wide variety of different configurations, all of which are contemplated herein.

FIG. 1 shows a front view of the nasal breathing device, where the three strips of medical-grade hypoallergenic adhesive tape that makes up the device are placed on the face as worn by a user. The three-inch by half-inch pieces of tape (101, 102) are placed above and below the lips. The three-inch by one-inch bridging piece of tape (100) is placed between the two strips mentioned previously (101, 102) and over the lips, holding the lips shut through hook and loop attachments

FIG. 2 shows a magnified view of the three strips that make up the device, where small vertical lines signify hook attachments and small loops signify loop attachments. Two strips (200, 202) have hook attachments and measure three inches long by half an inch wide. One strip (201) has loop attachments and measures three inches long by an inch wide.

FIG. 3 shows a side view of the device as it would be worn by a user. Two adhesive strips measuring three inches long by half an inch wide (300, 302) are placed above and below the lips, while the bridging strip measuring three inches long by an inch wide (301) is placed over the lips and the two previously mentioned strips, effectively holding the mouth shut.

FIGS. 4A-4B show a magnified view of the layers that make up the adhesive strips, where small horizontal lines signify hook attachments and small loops signify loop attachments. FIG. 4A shows a magnified view of an adhesive strip measuring three inches long by half an inch wide, where it is composed of a layer of adhesive (400), a carrier material (401), and hook attachments (402). FIG. 4B shows a magnified view of a bridging strip measuring three inches long by an inch wide, where it is composed of loop attachments (403) and a carrier material (404).

FIGS. 5A-5B show an embodiment of the device with electrodes embedded in it, from both front view and side view. FIG. 5A depicts two adhesive strips measuring three inches long by half an inch wide with electrodes, depicted by circles, embedded in the carrying material to be in contact with the skin of the user (500, 502), as well as the bridging strip measuring three inches by an inch wide (501). FIG. 5B depicts the same embodiment of the device as described in FIG. 5A, but from the side view, depicting how electrodes may be embedded in the adhesive strip material (503, 505), with no electrodes in the bridging strip (504).

FIG. 6 shows an embodiment of the device with multiple sensors embedded in the bridging adhesive strip. An alar pulse oximeter may be attached to the bridging strip and fastened to the nasal ala (600). A motion tracker may be attached to the bridging strip (601). A pulse tracker may be attached to the bridging strip (602). A respiration tracker may be attached to the bridging strip and fastened to the nasal septum (603).

FIG. 7 shows an embodiment of the device with a nasal dilator strip attached to the adhesive strip that lies above the upper lip. The nasal dilator strip may be attached to the adhesive strip that lies above the upper lip and below the nostrils so that it encircles the nose and runs across the bridge of the nose (701).

### DETAILED DESCRIPTION

Mouth breathing (also termed open-mouth breathing or mouth breathing habit) is breathing through the mouth rather than the nose. Human infants are sometimes considered obligate nasal breathers, but generally speaking healthy humans may breathe through their nose, their mouth, or both. During rest, breathing through the nose is common for most individuals. Breathing through both nose and mouth during exercise is also normal, a behavioral adaptation to increase air intake and hence supply more oxygen to the muscles. Mouth breathing may be called abnormal when an individual breathes through their mouth even during rest. Some sources use the term “mouth breathing habit” but this incorrectly implies that the individual is fully capable of normal nasal breathing, and is breathing through their mouth out of preference. However, in many cases, mouth breathing represents an involuntary, subconscious adaptation to reduced patency of the nasal airway, and mouth breathing is a requirement simply in order to get enough air.

Mouth breathing has been demonstrated to be associated with reduction of the retropalatal, retroglossal areas of the upper airway, and lengthening of the pharynx. The faster airflow generated by the longer and narrower upper airway may increase the negative intraluminal pressure during inspiration and thereby facilitate the collapse of the upper airway. (See: Lee et al: How does open-mouth Breathing Influence Upper Airway anatomy? Laryngoscope 117; June 2007.) Mouth breathing is associated with oral function as well. It promotes lip incompetence.

It promotes a lower tongue position and tongue thrusting with swallowing. It also promotes dry mouth and pharyngeal tissue. These desiccated tissues have increased inflammation, increased swelling and increased stickiness, and can also promote airway collapse and respiratory disturbance with increased effort.

It has been demonstrated during development of some of the embodiments of the present invention that unobstructed nasal breathing during sleep causes significantly fewer RERAs and lower RDIs than habitual mouth breathing during sleep.

A goal of treatment is, therefore, to promote an environment to breathe through the nose, if it is unobstructed. Deviated septums and enlarged turbinates may need to be evaluated and treated.

Many patients also suffer from sleep bruxism (SB). Sleep bruxism (SB) has historically been treated as an isolated oral issue. Recently however, research has shown a correlation between Sleep Bruxism (SB) and sleep arousals in patients with Sleep-Disordered Breathing (SDB). Sleep-Disordered Breathing (SDB) is defined as abnormalities in respiratory patterns or ventilation frequency during sleep. It pertains to 1) Upper Airway Resistance Syndrome, 2) Obstructive Sleep Apnea, and 3) Central Sleep Apnea. Hypoxia results from obstruction (a ventilatory problem) or improper respiratory pattern and blood gases/pH problems (an arousal problem).

Mouth breathing has been classified according to etiology into 3 groups: obstructive, habitual and anatomic. The nasal airway may be compromised partially, where there is increased resistance to the flow of air due to narrowing of the lumen at some point in the upper respiratory tract, or completely obstructed. Such individuals may find it difficult or impossible to breathe through their nose alone. Specific causes of nasal obstruction which have been linked to mouth breathing include antrochoanal polyps. Chronic mouth breathing in children may have implications on dental and facial growth. It also may cause gingivitis (inflamed gums) and halitosis (bad breath); especially upon waking if mouth breathing occurs during sleep.

“Pregnancy rhinitis” may lead to nasal obstruction and mouth breathing. This tends occur in the third trimester of pregnancy. In other cases, the upper lip may be short, and the lips do not meet at rest (“lip incompetence”). Gingivitis, gingival enlargement, and increased levels of dental plaque are common in persons who chronically breathe through their mouth. The usual effect on the gums is sharply confined to the anterior maxillary region, especially the incisors (the upper teeth at the front). The appearance is erythematous (red), edematous (swollen) and shiny. This region receives the greatest exposure to airflow during mouth breathing, and it is thought that the inflammation and irritation is related to surface dehydration, but in animal experimentation, repeated air drying of the gums did not create such an appearance.

It has been suggested that chronic mouth breathing in children can lead to the development of a long, thin face, sometimes termed “long face syndrome,” or specifically “adenoid faces” when the mouth breathing is related to adenoid hypertrophy. Malocclusion of the teeth (e.g. “crowded teeth”) is also suggested to result from chronic mouth breathing in children. Conversely, it has been suggested that a long thin face type, with corresponding thin nasopharyngeal airway, predisposes to nasal obstruction and mouth breathing, i.e., a long thin face may cause mouth breathing rather than the other way around. Facial form is also strongly influenced by genetic factors. The following other conditions are also associated with mouth breathing: cheilitis glandularis, Down syndrome, anterior open bite, tongue thrusting habit, cerebral palsy, sleep apnea and snoring.

Some individuals breathe through their mouth through force of habit, perhaps due to a previous cause of nasal obstruction that is now corrected. This is of significance to the present invention, as it has been found that simple occlusion of mouth breathing may cause the mouth-breather to revert back to normal nasal breathing patterns.

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present device may be embodied in other specific forms without departing from its spirit or characteristics. The described embodiments are to be considered in all respects only as illustrative and not restrictive. The scope of the embodiment is, therefore, indicated by the appended claims rather than by the foregoing description. All changes which come within the meaning and range of equivalency of the claims are to be embraced within their scope.

Unless otherwise defined herein, scientific and technical terms used in connection with the present embodiment shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present embodiment are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

The following terms and phrases, unless otherwise indicated, shall be understood to have the following meanings.

The present embodiment provides a device comprising hypoallergenic medical-grade adhesive.

“Hypoallergenic” in context of the embodiments herein may describe any object that cause fewer allergic reactions than its non-hypoallergenic counterpart, such as certain medical dressings made of materials known to provoke less allergic reactions than other materials. The term “hypoallergenic” as used herein describes materials that are known to contain a relatively small amount of allergens compared to other options.

The present embodiment provides a device using hook and loop attachments in order to connect approximately two half-inch wide pieces of adhesive across the lips through using an approximately inch-wide piece of adhesive.

“Hook and loop attachments” in context of the embodiments herein refer to two components together making up the singular object, for example, as taught by VELCRO®, wherein one piece is covered with tiny hooks, while the opposing piece is covered with smaller loops. When the two are pressed together, the hooks catch the loops and bind temporarily. The bond is easily broken and may be reapplied by pulling them apart and pressing them back together.

It has been found that occlusion of mouth breathing by the application of oral occlusion device to the lips during sleep may be helpful in causing a reversion to normal nasal breathing patterns. Such type of oral occlusion devices will be discussed below.

The product has a number of purposes, by way of example and not limitations only, include:

1. Promote a healthy breathing pattern in which inspiration and expiration occurs solely through the nose.
2. Detect problems with nasal restrictions and congestion.
3. Decrease sleep arousal frequency.
4. Reduce nocturnal sympathetic nervous system hyperactivity.
5. Reduce respiratory disturbances by improving respiratory effort in the effort to breath.

Benefits of use include, by way of example and not limitations only, include:

1. Improved airflow to the lungs as compared with mouth breathing.
2. Reduction of hyperventilation.
3. Hydration of the air entering the pharynx and lungs. This promotes less resistance to the airflow.
4. Prevention of dry mouth and its complications including gingivitis, cavities and halitosis.
5. Reduction in snoring.
6. Improvement in pulse oxygenation.
7. Reduction in sleep arousals, sleep bruxism, TMD pain, teeth pain and root canals, teeth wear.
8. Reduction in sympathetic tone and hypertension.
9. Reduction in asthma.
10. Reduction in cellular inflammation and associated chronic pain.
11. Reduction in oral developmental problems.
12. Reduction of reverse tongue position and swallowing problems.

An embodiment relates to a wearable device comprising two pieces of medical grade hypoallergenic adhesive tape measuring approximately three and a half inch long by approximately a half inch wide, and one piece of medical grade hypoallergenic adhesive tape measuring approximately three and approximately a half inch long by one inch wide; wherein the wearable device is configured to be worn with approximately two half inch-wide pieces above and below the user's lips, while approximately one inch-wide piece bridges the two other pieces across the user's lips; wherein the wearable device is configured to hold the user's lips shut, facilitating nasal breathing and reducing snoring and dry mouth.

In an embodiment, approximately two half inch-wide pieces of medical grade hypoallergenic adhesive above and below the lips have on one side adhesive to contour and adhere to the user's skin, and on the other side hook and loop attachments.

In an embodiment, approximately a inch-wide piece of medical grade hypoallergenic adhesive has on one side hook and loop attachments.

In an embodiment, the user's mouth is closed and nasal breathing is facilitated.

In an embodiment, the wearable device further comprises electrodes embedded in the strips above and below the lips that may electrically stimulate the orbicularis oris muscles to contract, closing the lips.

In the embodiment, the wearable device further comprises sensors embedded in the bridging strip above that measure oxygen saturation, respiration, sleep, and pulse as the user wears it.

In an embodiment, the wearable device further comprises an additional handheld device that may control the electrodes and the sensors.

In an embodiment, the wearable device further comprising a moisturizer, medicated ointment, gel, or other substance coating, permeating, or otherwise attached to the sides of approximately half inch-wide adhesive strips.

In an embodiment, the wearable device further comprises a nasal dilator strip attached to approximately a half inch-wide adhesive strip that lies above the upper lip.

In an embodiment, the wearable device further comprises differently colored adhesive strips, wherein the adhesive strips may be clear, skin tone, or decoratively patterned.

In an embodiment, the wearable device further comprises padding over the adhesive strips.

An embodiment relates to an oral occlusion apparatus for promoting nasal breathing, especially during sleep. Nasal breathing during sleep is desirable for many reasons, including but not limited to a better sleep, less snoring, and reduction of dry mouth. This device would provide optimum efficacy in preventing oral breathing while allowing the user to remove the device and replace it as they wish, improving quality of the experience.

Preferably the device is built from hypoallergenic, medical-grade adhesive tape. These materials can come into contact with skin for extended periods of time without being harmful to the user.

Preferably the device contains hook and loop attachments. These fasteners can be easily removed and replaced with minimal effort and still function effectively.

The device is largely made up of medical adhesive tape and hook and loop attachments.

In general, medical adhesive tape or bandage comprises a backing with two major surfaces, i.e., an outer major surface and an inner major surface, a reinforcing scrim, and a layer of pressure-sensitive adhesive disposed on the inner major surface thereof.

Examples of suitable polymers for the backing include thermoplastic polymers, preferably thermoplastic polymers that can be extruded (e.g., using a blown film or cast film extrusion process). Representative examples of thermoplastic polymers include polyolefins (e.g., low density polyethylene (LDPE), high density polyethylene (HDPE), linear low density polyethylene (LLDPE), polypropylene and polybutylene), polyester, copolyester, polyamide (e.g., nylon), polyvinyl chloride, polycarbonate, polytetrafluoroethylene, and mixtures thereof.

The scrim may be made of natural materials or synthetic materials. Illustrative examples of natural materials include cotton, silk, hemp, flax, and combinations thereof. Examples of synthetic materials include polyester, acrylic, polyolefin (e.g., polyethylene and polypropylene), nylon and combinations thereof. Natural materials and synthetic materials may also be combined, for example, in a 65/35 polyester/cotton blend or a 80/20 polyester/cotton blend.

The preferred pressure sensitive adhesives which can be used in the medical adhesive articles of the present invention are adhesives which are known to be useful for application to skin. The thickness of the adhesive layer is greater than is typically employed in medical tapes and bandages. Typically, the adhesive layer will be between at least about 20 grains per 24 square inches' area and about 40 grains per 24 square inches' area. Adhesive layers which are thinner than this range may tend to be more likely to leave residue when resultant medical articles are removed from a wearer whereas using thicker adhesive layers may tend to merely entail greater cost with no beneficial change in performance. A preferred class of adhesives are those disclosed in U.S. Pat. No. 6,441,092. One example is a blend of 85 weight percent of 2-ethylhexyl acrylate/acrylic acid/ABP (96.5/3.5/0.05 weight ratio) and 15 weight percent Avalure AC 210 Acrylate copolymer. Adhesives containing from about 5 to about 20 weight percent of such hydrophilic materials provide a good balance of desired moisture permeability without unduly softening the adhesive layer to yield undesirable levels of residue. These adhesives can provide a desirable “breathability” that permits pass through of sweat from the skin surface, making tapes and bandages of the invention more comfortable when worn in hot conditions. Other illustrative examples of useful adhesives include those described in U.S. Pat. No. 4,112,177 (particularly the tackified acrylate “skin layer adhesive” described in Example 1), U.S. Pat. No. 5,648,166, acrylate copolymers as described in U.S. Pat. No. RE 24,906, and a 70:15:15 isooctyl acrylate:ethyleneoxide acrylate:acrylic acid terpolymer, as described in U.S. Pat. No. 4,737,410 (see Example 31). Other illustrative examples of useful adhesives are described in U.S. Pat. Nos. 3,389,827, 4,112,213, 4,310,509, 4,323,557, and 6,497,949. If desired, medicaments or antimicrobial agents may be included in the adhesive, for example, as described in U.S. Pat. Nos. 4,310,509 and 4,323,557. The pressure sensitive adhesives preferably transmit moisture vapor to increase patient comfort. While moisture vapor transmission can be achieved through the selection of an appropriate adhesive, it is also contemplated in the present invention that other methods of achieving a high relative rate of moisture vapor transmission may be used, such as pattern coating the adhesive on the backing, as described in U.S. Pat. No. 4,595,001.

In the preferred embodiments according to the present invention, the choice of adhesives is limited to those that are safe to use on human or animal skin, and preferably to those that are of the class known as “hypoallergenic” adhesives. The preferred acrylate copolymers are adhesives of this class.

Medical adhesive tape is produced large-scale. First, a carrier material is chosen depending on the application required. The carrier material is a thermoplastic polymer including but not limited to polyolefins, polyester, nylon, or mixtures thereof. Once the proper carrier material is selected, it may be reinforced with a woven, knitted, or nonwoven scrim, which may be a natural or synthetic material, such as cotton, silk, polyester, nylon, or combinations thereof. A pressure-sensitive adhesive may then be added where the carrier material is directly coated with an acrylate-based adhesive. The large rolls produced in this fashion are then further processed into spools, cut, and packaged. [Source: U.S. Pat. No. 20070010777A1]

Hook and loop fasteners comprise mating strips or patches of filamentary stress-bearing hooks and loops. The hooks are woven, and the loops are knit or woven, into a textile backing, or ground. In order to secure the hook or loop elements to the ground, and to bond the fibers composing the ground to each other, in a manner to withstand the forces involved, the ground is impregnated with a resinous binder to form a composite structure. The hook and loop fasteners fabricated with various binders which improve the strength and durability of the fastener. In a first general aspect, a hook or loop component of a hook and loop fastener has a ground sheet and a pile of hook or loop elements extending from the ground sheet, and a solidified hot-melt binder of synthetic resin impregnating the ground sheet. Hot-melt binders which can used successfully in hook and loop fasteners contain the reaction product of free isocyanate groups which have entered into cross-linked bonds to effect cure of the binder. When moisture activates the cross-linking, the bonds are polyurethane-type bonds. Alternatively, exposing the resin to bifunctional or polyfunctional amine or alcohol yields ureido or urethane cross-link bonds, respectively. Hook and loop fasteners in which the binder is photo-cured, or in which cure is the result of free radical catalysis, are also useful. In other embodiments, hook and loop fasteners are fabricated with hot-melt, thermoplastic binders. Such thermoplastics include polyesters and polyamides. Binders which are composed of interpenetrating polymer networks may be used as well.

Hook and loop attachments, such as VELCRO®, are produced large-scale as well. Hook and loop attachments are formed through hot-melt manufacturing processes with thermoplastic binders. Thermoplastics include polyesters and polyamides, as well as binders composed of interpenetrating polymer networks. The pre-heated ground sheet is covered with a pile of hook or loop elements, while a liquid hot-melt binder of synthetic resin impregnates the ground sheet. The ground sheet is then cooled in an accumulator as it passes over rollers, and the adhesive may also be introduced to cure-accelerating agents at this time. [Source: U.S. Pat. No. 5,436,051.]

Hook and loop attachments may be manufactured attached to medical adhesive tape following standard industry procedures.

In one embodiment, the present device relates to an apparatus for nasal breathing, comprising three strips of medical-grade, hypoallergenic adhesive, wherein the device can be worn over the mouth and lips to prevent oral breathing.

The device of the present embodiment is advantageous over other devices on the market. Other devices created to promote nasal breathing for the large part either feature tape or adhesive holding the lips shut, or a facial mask that covers the lips and nose. Both of these designs are not made to be easily removed and replaced within the same session of use. In one embodiment, the device of the present embodiment overcomes this limitation.

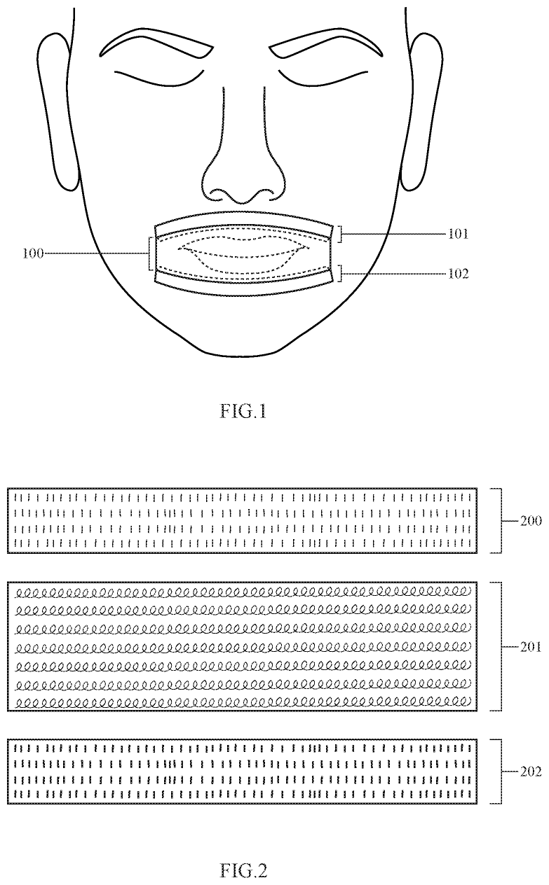

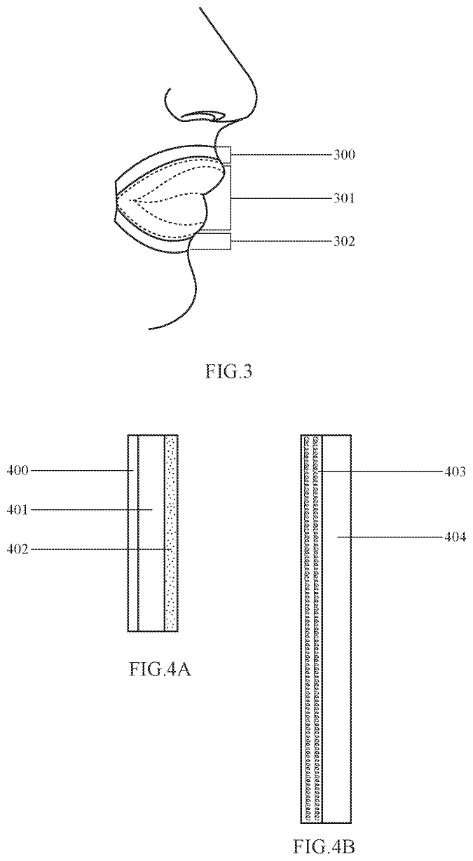

In another aspect, an embodiment relates to a device for promoting nasal breathing as shown in FIGS. 1 and 3, comprising three strips of medical-grade hypoallergenic adhesive tape, wherein two approximately three inch long by approximately half an inch wide strips of tape are placed above and below the lips (101 and 102, 300 and 302), while one approximately three inch long by approximately one inch wide strip of tape (100, 301) is placed between the two strips over the lips, bridging the two and holding the lips shut through hook and loop attachments. In one embodiment, the non-adhesive strip of tape i.e., the bridging strip (100, 301) may further comprise a low-friction surface that allows the lips to move more freely when the user intends to open the mouth to breathe, cough or speak. The non-adhesive strip of tape (100, 301) can be optionally made of elastic materials with various thickness and flexibility. Potential materials of the non-adhesive strip of tape (100, 301) include polyurethane (PU), polyethylene (PE), silicone, cloth, non-woven cloth, PET, paper, as well as other flexible thin films. When the user intends to open the mouth by exerting force on muscles around the lips or on the lateral pterygoid muscles to depress the mandible (lower jaw), the elastic non-adhesive band is extended to a height larger than its original height. This elastic non-adhesive strip of tape (100, 301), if the mouth is opened creates a flow passage within the non-obstructive mouth opening area allows the user to cough or speak more freely. When the user relaxes (without exerting force on muscles), the elastic non-adhesive band will return to its original height and keep the mouth in closed condition again.

In an embodiment, the bridging strip will self-reposition and talking and coughing are possible without having to remove the bridging strip. Elastic materials may be stretched by a certain percent beyond their relaxed length and these materials will recover to substantially their original relaxed length upon release of the stretching force and thus self-reposition themselves.

In another embodiment, the non-adhesive strip of tape i.e., the bridging strip (100, 301) can be made of relatively non-elastic material with little or no flexibility. In this embodiment, the user has to remove the strip if the user intends to open the mouth or to cough or to speak more freely. Non-elastic material limit stretching of the non-adhesive strip of tape i.e., the bridging strip (100, 301). For example, in embodiments the non-adhesive strip of tape i.e., the bridging strip (100, 301) is a wool, cloth, or similar material. The non-adhesive strip of tape i.e., the bridging strip (100, 301) may be a fibrous material, such as a low-stretch polyethylene, polyamide, polyester, polyether, siloxane, or other material. In an embodiment, the non-adhesive strip of tape i.e., the bridging strip (100, 301) may be a strip of TYVEK® HDPE film. The non-adhesive strip of tape i.e., the bridging strip (100, 301) may extend across the entire inner surface, or only along a portion of the inner surface. In certain embodiments, it may be desirable for the non-elastic strip to extend across between approximately 25 and 100%, such as approximately 25, 50, or 75% of a length of the inner surface to reduce the overall elasticity of the non-adhesive strip of tape i.e., the bridging strip (100, 301). The non-elastic strip may be substantially centered along the length, or may be offset toward either of the first or second ends perpendicular to the adhesive tapes or strips that are placed above and below the lips (101 and 102, 300 and 302). In all the above embodiments, the material chosen preferably is lightweight and durable, water-resistant yet breathable, repels water and intact that can resist repeated folding and flexing without tearing and safe for using in close contact with skin. The material chosen preferably is suitable for manufacturing operations such as gluing, lamination, sewing, stapling of the Velcro.

As shown in FIG. 2, the two strips surrounding the lips may have a surface comprising the hook side of the hook and loop attachments (200 and 202), while the bridging strip may comprise the loop side of the hook and loop attachments (201).

As shown in FIG. 4, the two strips surrounding the lips may be comprised of three basic layers, where adhesive (400), a carrier material (401), and hook and loop attachments (402) are layered, with adhesive directly in contact with the user's skin and the hook and loop attachments facing up. The bridging strip may be comprised of two basic layers, where a first layer (403) comprises hook and loop attachments, and a second layer (404) comprising a carrier material. In one embodiment, the adhesive may be a reusable hydrogel adhesive. In one embodiment, the layer of adhesive that comes into contact with the user's skin may be permeated, coated with, or otherwise carrying a medicated ointment, gel, or other substance known to those skilled in the art that may be absorbed by the skin. In one embodiment, the layer of carrier material may be padded in order to provide greater comfort to the user.

In one aspect of this embodiment, the device is suitable for promoting nasal breathing in order to provide a better night's sleep.

In one aspect of this embodiment, the device is suitable for promoting nasal breathing in order to prevent snoring.

In another aspect of this embodiment, the device is suitable for promoting nasal breathing in order to help prevent dry mouth.

In another aspect of this embodiment, the device is suitable for promoting nasal breathing in order to help combat sleep apnea.

Another embodiment features reusable versions of the upper and lower lip strips, allowing them to be taken off and reapplied multiple times without decreasing in efficacy. The reusable adhesive is similar in function to those found in EKGs and TENS units, being a medical-grade hydrogel adhesive. The adhesive may be attached to the medical tape composing the upper and lower strips, coming directly in contact with the user's skin. The adhesive may be in the form of a gel pad impregnated in a porous matrix and adherent to the body surface, being sufficiently cohesive to cling to the user's skin. The adhesive may be manufactured according to industry standard in the respective field as taught in US Pat. No. 4,871,490A, where synthetic or natural polymers are crosslinked, conditioned, and sterilized in order to form a flexible, bacteria-resistant adhesive gel, or in any other method known to those skilled in the art.

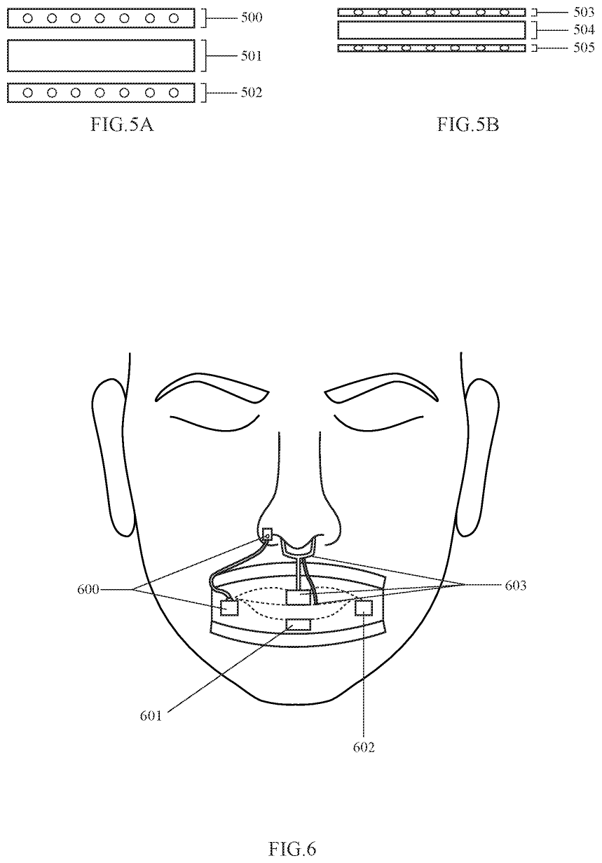

Another embodiment shown in FIG. 5 features small wireless electrodes attached to the strips surrounding the lips directly on the skin that may be turned on and off in order to stimulate the orbicularis oris muscles to contract (500-502). This allows the user to utilize them for training lip closure and nasal breathing, as well as reducing snoring. Electrodes may be embedded directly into the adhesive strips lying above and below the lips in order to lie directly on the skin (503-505). The electrodes may be manufactured according to industry standard, where an electrically conductive gel is attached to an electrically conductive metallic layer and may administer electrical stimulation, as taught by U.S. Pat. No. 6,845,272B1. As electrodes gently stimulate the muscle they are placed on, the muscle contracts until the electrical current is removed. The user may use this electrical current to control muscle contraction to keep their mouth closed and facilitate nasal breathing while they are asleep in order to train lip closure.

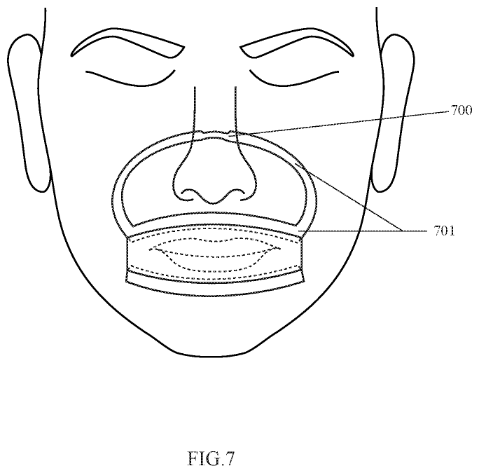

Another embodiment features multiple sensors attached to the bridging strip that include a pulse oximeter, a respiration tracker, a sleep tracker, and a pulse tracker in order to monitor the user's health and vitals as they sleep, as depicted in FIG. 6. An alar pulse oximeter (600) attached to the bridging strip can be fastened to the nasal ala in order to monitor a branch of the internal carotid artery and detect changes in oxygen saturation as the user sleeps. Through measuring the amount of light absorbed by the skin above the vein, the pulse oximeter can determine the oxygen saturation in the user's bloodstream, which is desirable if the user has sleep apnea or another respiratory issue that prevents them from obtaining the oxygen they need from the air. As to the specific area of the nasal septum that is preferred for use of a nasal pulse oximeter probe, it has been learned that the area of the nasal septum closest to the face (e. g., the proximal area of the middle alar), is more consistently vascularized and thereby provides more consistent and reliable signals than the areas more distal, i. e., the septum closer to the point of the nose. In particular, and more specifically, a highly vascularized region of the septum known alternately as Kiesselbach's plexus and Little's area, is a preferred target area for detection of blood oxygen saturation levels by a nasal pulse oximeter probe of the present invention. A respiration tracker (603) attached to the bridging strip can track breaths per minute through the use of oronasal thermal sensors with the ability to measure fluctuations in temperature with respiration, which is also desirable if the user has sleep apnea or another respiratory issue that prevents them from breathing evenly and consistently throughout the night. In an embodiment, of the invention, the integrated sensor is a printed electrically-conductive ink thermistor also interchangeably referred to herein as a line sensor. Conductive ink changes resistance with changes in temperature. Exhalation heats the ink and inhalation cools the ink. The changing temperature of the conductive ink can be recorded by noting the respective changes in electrical resistance of the conductive ink line. One exemplary type of conductive ink suitable to be used as a thermistor is carbon ink DS 119-28 manufactured by Creative Materials, Inc., Mass., USA. Carbon ink 119-28 is an extremely flexible, pad printable, electrically, conductive, carbon filled ink, suitable for application by pad printing, dipping and syringe dispensing. This product features excellent adhesion to Kapton, Mylar, glass and a variety of other surfaces. Unlike many other conductive materials, this product is very resistant to flexing and creasing. Any such conductive inks or conductive elastomers with similar attributes as those enumerated above are included within the scope of the invention. The conductive ink does not come into contact with the skin of a user and therefore does not require either insulation or skin contact biocompatibility. Of course, insulation can be added where desired. A sleep tracker is also attached that utilizes a motion sensor (601) built into the bridging strip, where a sensor fixed over the user's lips tracks movement as they sleep and can extrapolate based on movement if their sleep was disturbed by an apneic fit or other episode. A pulse tracker (602) is also attached to the bridging strip which measures heart rate by either using light to measure the blood volume controlled by the heart's pumping action, or through measuring the bio-potential generated by electrical signals controlling the expansion and contraction of cardiac muscles. This is also desirable in cases of disturbed sleep or breathing, where it is important to track pulse rate.

Another embodiment includes a handheld device to be used in adjusting electrode stimulation (specifically force, frequency, and duration of the contraction) in order to tailor the device's function to the user's specific needs. The device may also track all vitals listed above and provide alerts for decreased respiration and/or dropping oxygen saturation. Through wireless communication with the electrodes and/or the sensors named above, the device can track data at a pre-determined rate and store the data overnight or for a specified duration of time in order to analyze patterns or track changes.

Another embodiment features moisturizer, ointment, gel, or other substance on the adhesive strips directly in contact with the skin in order to provide relief to dry or cracked skin, or improve comfort. Substances used include but are not limited to lubricants, antibiotics, drugs, flavorings, silicone gel, or other substances known to those skilled in the art. The substance may be infused, injected, permeated, printed, coated, sprayed, soaked, baked, seared, or otherwise attached or integrated with the medical adhesive. The substance may be in liquid gel, powder, capsule, crystalline, or other forms for absorption into and through the tissues and organs of the body, as taught by US Pat. No. US20150217098A1. The substance within the tape adhesive may be diffused transdermally while being worn, thereby having a therapeutic effect on the user.

Another embodiment includes an attached external adhesive nasal dilator strip (700) in order to gently expand the nasal wall tissue, thereby further facilitating nasal breathing. The nasal dilator strip, e.g., as done by BREATHE RIGHT STRIPS™. The nasal dilator strip comprises a flat flexible strip member with an inner surface and an outer surface, and an adhesive bandage, having an adhesive surface and a non-adhesive surface, securing the outer surface of the flexible strip member on to the adhesive surface of the adhesive bandage, thereby temporarily securing the flexible strip member to the bandage, which is attached perpendicular to the user's nose as taught by US Pat. No. US20130190807A1. The nasal dilator strip may be attached to the adhesive strip of the device that lies above the upper lip so that it encircles the nose and runs across the bridge of the nose (701), as shown in FIG. 7.

In another embodiment, the device may be formed of a clear material. Alternatively, the device may be neutral or skin toned. Alternatively, the device may have a decorative pattern or color.

In another embodiment, the device may include padding in the three tape strips in order to facilitate ease of use while sleeping by those who sleep on their sides or stomach. The tape comprising the device may comprise a first layer having a bottom and top surface, where the bottom surface is an adhesive skin-contacting surface that adheres to the skin, and a second layer on top of the first layer comprising a layer of padding, as taught by U.S. Pat. No. 9,986,772. In the manufacturing of the device enclosed herein, a third layer is added comprising one side of the hook and loop attachments used. Having illustrated the present device, it should be understood that various adjustments and versions might be implemented without venturing away from the essence of the present invention. Further, it should be understood that the present device is not solely limited to the device as described in the embodiments above, but further comprises any and all embodiments within the scope of this application.

All references, including granted patents and patent application publications, referred herein are incorporated herein by reference in their entirety.

1. A wearable device comprising an adhesive strip and a bridging strip, the adhesive strip comprising a medical-grade hypoallergenic adhesive that is configured to be adhered to a face of a human or an animal at locations above and below lips of the human or the animal, wherein the bridging strip is configured to be placed over the lips so as to bridge the adhesive strip and hold the lips shut via a hook and loop attachment, wherein the hook and loop attachment is configured to permit removing and reattaching of the bridging strip for a plurality of times without loss of ability to hold the lips shut, wherein the wearable device is configured to hold the lips shut and promote nasal breathing.

2. The wearable device of claim 1, wherein the adhesive strip comprises a single piece of adhesive strip or a plurality of adhesive strips, and the adhesive strip has a first side and a second side, wherein the first side comprises the medical-grade hypoallergenic adhesive and the second side comprises at least a hook portion or a loop portion of the hook and loop attachment.

3. The wearable device of claim 1, wherein the bridging strip comprises at least a hook portion or a loop portion of the hook and loop attachment.

4. The wearable device of claim 1, further comprising an electrode embedded in the adhesive strip.

6. The wearable device of claim 4, wherein the electrode is configured to electrically stimulate an orbicularis oris muscle to contract and close the lips.

7. The wearable device of claim 1, further comprising a sensor embedded in the bridging strip.

8. The wearable device of claim 6, wherein the sensor is configured to measure oxygen saturation, respiration, sleep, and pulse.

9. The wearable device of claim 1, wherein at least one of the adhesive strip comprise a moisturizer, a medicated ointment, a medicated gel, a medicated coating.

10. The wearable device of claim 1, wherein at least one of the adhesive strip comprise a nasal dilator strip.

11. The wearable device of claim 1, wherein the adhesive strip have a color and/or a pattern.

12. The wearable device of claim 10, wherein the color comprises clear or skin tone, and the pattern comprises a decorative pattern.

13. The wearable device of claim 1, further comprising a padding over the adhesive strip.

14. A system comprising a wearable device and a handheld device, the wearable device comprising an adhesive strip, a bridging strip, an electrode embedded in the adhesive strip, and a sensor embedded in the bridging strip, wherein the adhesive strip comprises a medical-grade hypoallergenic adhesive that is configured to be adhered to a face of a human or an animal at locations above and below lips of the human or the animal, wherein the bridging strip is configured to be placed over the lips so as to bridge the adhesive strip and hold the lips shut via a hook and loop attachment, wherein the wearable device is configured to hold the lips shut, and wherein the handheld device is configured to control the electrode.

15. The system of claim 13, wherein the electrode is configured to electrically stimulate an orbicularis oris muscle to contract and close the lips, facilitating nasal breathing and reducing snoring and dry mouth.

16. The system of claim 13, wherein the sensor is configured to measure oxygen saturation, respiration, sleep, and pulse.

17. A method comprising obtaining a wearable device and attaching the wearable device to a face of a human or an animal, wherein the wearable device comprises an adhesive strip and a bridging strip, the adhesive strip comprising a medical-grade hypoallergenic adhesive that is configured to be adhered to the face of the human or the animal at locations above and below lips of the human or the animal, wherein the bridging strip is configured to be placed over the lips so as to bridge the adhesive strip and hold the lips shut via a hook and loop attachment, wherein the wearable device is configured to hold the lips shut and promote nasal breathing.

18. The method of claim 16, wherein the adhesive strip has a first side and a second side, wherein the first side comprises the medical-grade hypoallergenic adhesive and the second side comprises at least a hook portion or a loop portion of the hook and loop attachment.

19. The method of claim 16, wherein the bridging strip comprises at least a hook portion or a loop portion of the hook and loop attachment.

20. The method of claim 16, further comprising an electrode embedded in the adhesive strip, wherein the electrode is configured to electrically stimulate an orbicularis oris muscle to contract and close the lips.

21. The method of claim 16, further comprising a sensor embedded in the bridging strip, wherein the sensor is configured to measure oxygen saturation, respiration, sleep, and pulse.
